(19) United States
(12) Patent Application Publication
(10) Pub. No.: US 2023/0178253 A1
(43) Pub. Date: Jun. 8, 2023

(54) MACHINE LEARNING METHOD FOR IDENTIFYING DRUG INTERACTIONS

(71) Applicant: Insight Direct USA, Inc., Tempe, AZ (US)

(72) Inventors: Michael Griffin, Wayland, MA (US); Hailey Kotvis, Sussex, WI (US); Josephine Miner, Scituate, RI (US); Kayla Poulsen, Natick, MA (US); Katherine Lumb, Ponte Vedra, FL (US)

(21) Appl. No.: 17/696,498

(22) Filed: Mar. 16, 2022

Related U.S. Application Data
(60) Provisional application No. 63/286,828, filed on Dec. 7, 2021.
(60) Provisional application No. 63/313,494, filed on Feb. 24, 2022.

(51) Int. Cl. G16H 70/40; G16H 50/20; G16H 50/70

(52) U.S. Cl. CPC G16H 70/40; G16H 50/20; G16H 50/70

(57) ABSTRACT

A method for identifying drug interactions that occur between various drugs utilized to treat various morbidities. Each drug combination used to treat a morbidity has an efficacy that can be measured relative to a baseline real world efficacy. Differences between effective and ineffective drug combinations are identified and drug interactions are identified based on overlapping drugs in the effective and ineffective combinations.

### CROSS-REFERNCE TO RELATED APPLICATION(S)

This application claims priority to U.S. Provisional Application No. 63/286,828 filed Dec. 7, 2021, and entitled “METHOD FOR DETERMINING THE EFFECTIVENESS OF VARIOUS TREATMENT ACTIONS AND GENERATING PATIENT-SPECIFIC TREATMENT ACTIONS BASED ON THE EFFECTIVENESS,” and claims priority to U.S. Provisional Application No. 63/313,494 filed Feb. 24, 2022, and entitled “A MACHINE LEARNING METHOD FOR DETERMINING THE EFFICACY OF VARIOUS TREATMENT ACTIONS AND GENERATING PATIENT-SPECIFIC TREATMENT RECOMMENDATIONS AND IDENTIFYING DRUG INTERACTIONS,” the disclosures of which are hereby incorporated by reference in their entireties.

### BACKGROUND

This disclosure relates generally to predictive healthcare. More specifically, this disclosure relates to enhancing patient and provider actions using machine learning models.

Patient interactions with healthcare systems are commonly known as encounters. Encounters may be office visits, prescriptions, nurse visits, text messages, phone calls, billings, surgeries, and the like. Predictive models are becoming more and more common in healthcare settings, but, by themselves, they do not predict treatment decisions to benefit patient health. Healthcare providers are required to interpret predictive results and other patient data in order to make treatment decisions. Because of the vast and ever-increasing number of treatment options and despite years of training and experience, healthcare providers often make suboptimal decisions, either with respect to patient health, healthcare costs, or both.

In general, it can safely be assumed that medications approved by governmental agencies (e.g., the U.S. Food and Drug Administration (FDA)) are effective at treating the diseases for which they are prescribed. However, many patients do not have their diseases controlled despite being prescribed these medications. There are many reasons patients may not improve, but possibly the most common reason is that patients do not take their prescribed medications. This is called prescription non-adherence. Uncomfortable and/or severe medication side effects contribute to up to 35% of patients being prescription non-adherent. In addition to the negative impact on patient health, non-adherence leads to billions of dollars in avoidable medical spending annually.

It is common practice to prescribe two or more medications to treat a disease. Hypertension, for example, is often treated with two to five medications from nine different medication classes. Many patients are prescribed medications for multiple diseases. Each of these medications can produce side effects in the patients who take them. Often patients are prescription non-adherent because the side effects from the medications are too uncomfortable or make it difficult to conduct normal daily activities, such as driving a vehicle.

All medications have side effects, some of which can be quite uncomfortable. Many medications have dozens of side effects. Most diseases are treated with multiple medications, which means that patients with a single disease will be subjected to many dozens of side effects. Many patients have multiple diseases and are prescribed even more medications, which means those patients can potentially be subjected to hundreds or thousands of side effects. Many medications also interact with each other in negative ways; for example, side effects from one medication may cause another medication to be less effective. Moreover, a single side effect caused by two or more medications can be additive such that a usually benign side effect is magnified to the point of severity. For example, two medications that both cause mild nausea when taken separately may cause severe nausea when taken together. One of the main reasons patients become prescription non-adherent is due to the negative interactions of the medications they have been prescribed. While uncommon, the side effects of one medication can minimize the severity of the side effects of other medications, i.e., two or more medications may positively interact (e.g., one medication causes excess production of saliva and another causes dry mouth).

Medications prescribed to treat disease are subject to rigorous clinical trials before being approved for use. This approval implies that all medications are effective at treating the diseases for which they are prescribed. This approval does not imply that the medication has been shown to be effective when prescribed in combination with other medications. Clinical trials may discover some small number of medication interactions, but it is not practical and is cost prohibitive to consider all but a small fraction of possible interactions.

The current state-of-the-art is to follow FDA-approved guidelines when prescribing medications to a patient. These guidelines are sets of rules based on the aggregate response of patient populations to the medications during clinical trials. Because guidelines are based on aggregations of patients, they are not patient-specific. However, every patient is different and responses to medications can vary greatly even for seemingly very similar patients. The guidelines are also based on the results of carefully controlled clinical trials where adherence is monitored, i.e., clinical trials may not accurately reflect non-adherence in practice. Due to time and financial constraints, clinical trials cannot account for every possible side effect and drug combination. Clinical trials may not quantify the severity of side effects, which may make it difficult for clinicians and pharmacists to assess medication combinations that lead to non-adherence.

Current technologies focus on the prevention of prescription errors to minimize injury, as opposed to preemptively prescribing a medication most suitable for each specific patient. These technologies and guidelines also do not provide individualized treatments for individual patients.

### SUMMARY

According to one aspect of the present disclosure, a method of generating personalized health treatment recommendations includes receiving, by a machine learning model trained to identify an efficacy of a plurality of drug combinations in treating the subject morbidity based on baseline health data and implemented on a treatment evaluator having memory and control circuitry, pertinent health data for a subject patient having a subject morbidity, wherein the baseline health data is generated based on sets of features extracted from sets of electronic medical records of each patient of a patient population associated with the subject morbidity and labeled as corresponding to either a positive outcome or a negative outcome with respect to the subject morbidity; classifying, by the machine learning model, the plurality of drug combinations as effective or ineffective based on the pertinent health data; and outputting, by the treatment evaluator, a first drug combination of the plurality of drug combinations as a candidate treatment for the subject patient based on the first drug combination of the plurality of drug combinations being classified as effective by the machine learning model.

According to an additional or alternative aspect of the present disclosure, a method of generating personalized health treatment recommendations includes receiving, by a machine learning model trained to identify an efficacy of a plurality of drug combinations in treating a subject morbidity based on baseline health data and implemented on a treatment evaluator having a memory and control circuitry, pertinent health data for a subject patient having the subject morbidity, the pertinent health data based on electronic medical records of the subject patient and including at least one patient-specific side effect, wherein the baseline health data is generated based on set of features extracted from sets of electronic medical records of each patient of a patient population associated with the subject morbidity and labeled as corresponding to either a positive outcome or a negative outcome with respect to the subject morbidity; receiving, by the machine learning model, side effect information regarding each drug of the plurality of drug combinations; classifying, by the machine learning model, each drug combination of the plurality of drug combinations as effective or ineffective based on the pertinent health data and the side effect information; and outputting, by the machine learning model, a first drug combination of the plurality of drug combinations as a candidate treatment for the subject patient based on the first drug combination of the plurality of drug combinations being classified as effective.

According to another additional or alternative aspect of the present disclosure, a method of identifying drug interactions includes generating, by a machine learning model trained to identify an efficacy of a plurality of drug combinations in treating a subject morbidity based on baseline health data and implemented on a treatment evaluator having a memory and control circuitry, efficacy data for each drug combination of the plurality of drug combinations, wherein the baseline health data is generated based on set of features extracted from sets of electronic medical records of each patient of a patient population associated with the subject morbidity and labeled as corresponding to either a positive outcome or a negative outcome with respect to the subject morbidity; comparing a first drug combination of the plurality of drug combinations to a second drug combination of the plurality of drug combinations to identify a cross-over drug present in both the first drug combination and the second drug combination; and classifying a first drug present in the second drug combination and not present in the first drug combination as interacting with the cross-over drug present in both the first drug combination and the second drug combination, wherein the efficacy data indicates that the first drug combination has a different efficacy than the second drug combination.

According to yet another additional or alternative aspect of the present disclosure, a method of identifying drug interactions includes generating, by a machine learning model trained to identify an efficacy of a plurality of drug combinations in treating a subject morbidity based on baseline health data and implemented on a treatment evaluator having a memory and control circuitry, efficacy data for each drug combination of a plurality of drug combinations present in the baseline health data for treating the subject morbidity, wherein the baseline health data is generated based on sets of features extracted from sets of electronic medical records of each patient of a patient population associated with the subject morbidity and labeled as corresponding to either a positive outcome or a negative outcome with respect to the subject morbidity; identifying, by the machine learning model, a first subset of drug combinations of the plurality of drug combinations as effective and a second subset of drug combinations of the plurality of drug combinations as ineffective; comparing the first subset of drug combinations to the second subset of drug combinations to identify cross-over drugs present in both a first drug combination of the first subset of drug combinations and a second drug combination of the second subset of drug combinations; classifying a first drug present in the second drug combination and not present in the first drug combination as adversely interacting with the cross-over drugs present in both the first drug combination and the second drug combination; and outputting, by the treatment evaluator, interaction data regarding the first drug, the interaction data indicating that the first drug adversely interacts with the cross-over drugs.

### BRIEF DESCRIPTION OF THE DRAWINGS

FIG. 1 is a block diagram of a treatment evaluator.

FIG. 2 is a flowchart illustrating a method of generating personalized health recommendations.

FIG. 3 is a flowchart illustrating a method of generating personalized health recommendations.

FIG. 4 is a flowchart illustrating a method of generating personalized health recommendations.

FIG. 5A is a flowchart illustrating a method of identifying and classifying drug interactions.

FIG. 5B is illustrates a drug interaction table.

### DETAILED DESCRIPTION

The present disclosure combines machine learning techniques with optimization techniques in a software system for providing individualized and predictive patient healthcare. Data from patient electronic medical records (EMRs) (e.g., medical records, insurance records, prescription records, and any other form of electronically stored health information) is used to train a number (for example, tens, hundreds, or thousands) of machine learning models to predict patient responses to various treatment actions. The machine learning models can be referred to collectively as a treatment machine learning model. Baseline health data is generated based on the EMRs and that baseline health data is used to train the treatment machine learning model to identify drug combinations that are effective at treating one or more morbidities.

The treatment machine learning model is configured to determine the efficacy of the treatment actions. The treatment machine learning model can, in some examples, identify treatment actions, such as drugs and combinations thereof prescribed to a patient, as being one of effective and ineffective at treating one or more target or subject morbidities. In some examples, the efficacy data generated by the treatment machine learning model can include information relating to both positive and negative interactions between the drugs forming the drug combinations. In some examples, the treatment machine learning model can determine the most effective set of medications for an individual patient by determining the set of interactions (e.g., side effects) most likely to be tolerated by each patient. The machine learning model can identify one or more sets of medications that will treat the patient’s morbidities and maximize the likelihood that the patient will be prescription adherent. Techniques according to the disclosure can deliver personalized medication recommendations based on predictions of individual patient tolerance for interactions, rather than generic guidelines based on patient populations.

In one example, the software system of the present disclosure can generate treatment recommendations that are likely to lead to prescription adherence. Side effects of various medications can be identified and the treatment machine learning model can generate candidate treatments based on the side effect information. Each patient may have a different tolerance for different side effects. Specific side effects can be identified as more or less tolerable for that specific patient depending on the needs of that specific patient, which information can be referred to as treatment tolerance information. The treatment tolerance information thus includes at least one patient-specific side effect. It is understood that the patient-specific side effect can be positive (e.g., currently has excess saliva so wants dry mouth) or negative (e.g., blurry vision when employed as a driver).

In some examples, the treatment tolerance information can be generated based on EMRs of the specific patient. For example, a patient can be classified as less tolerant to blurred vision based on the patient being employed as a driver, the patient living in a remote area, the patient living alone, the patient indicating intolerance, etc. A patient can be classified as more tolerant to blurred vision based on the patient living with other people, having assistance, the patient indicating tolerance, etc. In some examples, the treatment machine learning model can be configured to generate the treatment tolerance information based on the sets of EMRs for the patient population.

Techniques according to the present disclosure can identify effective and ineffective drug combinations and determine interactions between drugs forming the drug combinations about which little is known. Such information can be used to guide current and future drug development. For example, techniques according to the disclosure can identify certain drug combinations that have been effective in treating disease A. Similar but slightly different combinations are determined to be counterproductive in treating disease A. Techniques according to the disclosure can be used to understand the differences between these combinations based on the efficacy at treating the one or more diseases. The drug interaction data can be used to guide drug development to, for example, design new drugs that work better with popular combinations of existing drugs.

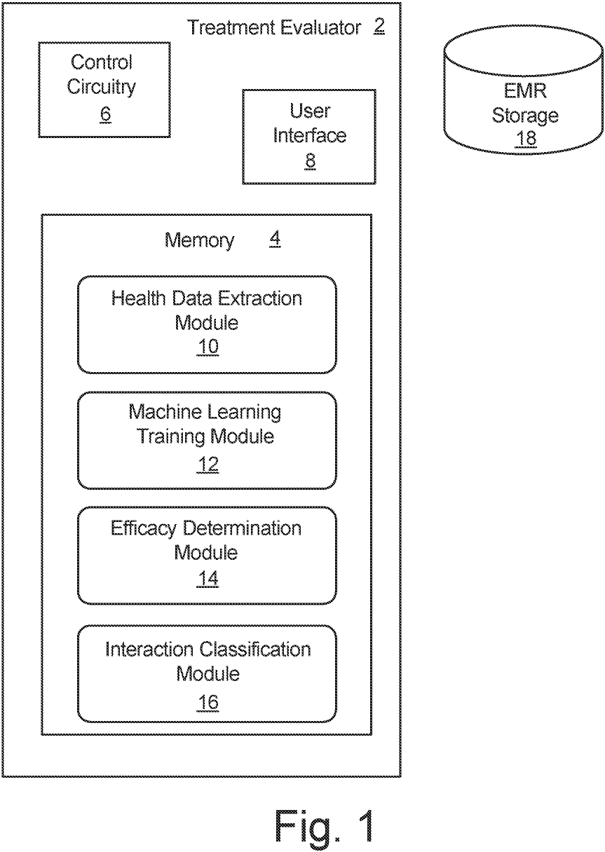

FIG. 1 is a block diagram illustrating treatment evaluator 2. Treatment evaluator 2 includes memory 4, control circuitry 6, and user interface 8. Treatment evaluator 2, which can also be referred to as a computing device, is configured to implement one or more treatment machine learning models trained on baseline health data to generate and output treatment information for patients. In some examples, the treatment evaluator 2 can be configured to generate information regarding various drug combinations utilized to treat a subject morbidity. Each drug combination can include one or more drugs (e.g., medications, supplements, etc.). In some examples, the treatment evaluator 2 is configured to generate and output candidate treatment combinations that are determined by the treatment machine learning model to improve patient outcomes by increasing prescription adherence. In some examples, the treatment evaluator 2 is configured to generate and output interaction data regarding interactions between various drug combinations used to treat the subject morbidity. Negative and positive interactions between various drugs forming the drug combinations can be identified based on the generated interaction data. The memory 4 can store health data extraction module 10, machine learning training module 12, efficacy determination module 14, and interaction classification module 16.

The treatment evaluator 2 is configured to generate data and information regarding predictive prescription adherence and/or information regarding proposed treatments for a patient and/or information regarding drug interactions. The treatment evaluator 2 is configured to store software, implement functionality, and/or process instructions. The treatment evaluator 2 can be of any suitable configuration for gathering data, processing data, etc. The treatment evaluator 2 can receive inputs, provide outputs, generate efficacy and/or interaction data and output information regarding predictive treatment actions for enhancing (e.g., optimizing) patient health. The treatment evaluator 2 can be configured to receive inputs and/or provide outputs via a user interface 8. The treatment evaluator 2 can include hardware, firmware, and/or stored software. The treatment evaluator 2 can be entirely or partially mounted on one or more circuit boards.

The treatment evaluator 2 can be a discrete assembly or be formed by one or more devices capable of individually or collectively implementing functionalities and generating and outputting data as discussed herein. The treatment evaluator 2 can be considered to form a single computing device even when distributed across multiple component devices. The treatment evaluator 2 is configured to perform any of the functions attributed herein to the treatment evaluator 2, including receiving an output from any source referenced herein, detecting any condition or event referenced herein, and generating and providing data and information as referenced herein. The treatment evaluator 2 can be of any type suitable for operating in accordance with the techniques described herein. In some examples, the treatment evaluator 2 can be implemented as a plurality of discrete circuitry subassemblies. In some examples, the treatment evaluator 2 can include or be implemented at least in part as a smartphone or tablet, among other options. In some examples, the treatment evaluator 2 can include and/or be implemented as downloadable software in the form of a mobile application. The mobile application can be implemented on one or more computing devices, such as a personal computer, tablet, and/or smartphone, among other suitable devices.

The treatment evaluator 2 can include control circuitry 6, memory 4, and the user interface 8. The control circuitry 6, in one example, is configured to implement functionality and/or process instructions. For example, the control circuitry 6 can be capable of processing instructions stored in the memory 4. Examples of control circuitry 6 can include one or more of a processor, a microprocessor, a controller, a digital signal processor (DSP), an application specific integrated circuit (ASIC), a field-programmable gate array (FPGA), or other equivalent discrete or integrated logic circuitry. The control circuitry 6 can be entirely or partially mounted on one or more circuit boards.

The memory 4 of the treatment evaluator 2 can be configured to store data and information before, during, and/or after operation. The memory 4, in some examples, is described as computer-readable storage media. In some examples, a computer-readable storage medium can include a non-transitory medium. The term “non-transitory” can indicate that the storage medium is not embodied in a carrier wave or a propagated signal. In certain examples, a non-transitory storage medium can store data that can, over time, change (e.g., in RAM or cache). In some examples, the memory 4 is a temporary memory, meaning that a primary purpose of the memory is not long-term storage. The memory 4, in some examples, is described as volatile memory, meaning that the memory 4 does not maintain stored contents when power to the treatment evaluator 2 is turned off. Examples of volatile memories can include random access memories (RAM), dynamic random access memories (DRAM), static random access memories (SRAM), and other forms of volatile memories. In some examples, the memory 4 is used to store program instructions for execution by the control circuitry 6. The memory 4, in one example, is used by software or applications running on the treatment evaluator 2 (e.g., by one or more computer-implemented machine learning models) to temporarily store information during program execution.

The memory 4, in some examples, also includes one or more computer-readable storage media. The memory 4 can be configured to store larger amounts of information than volatile memory. The memory 4 can further be configured for long-term storage of information. In some examples, the memory 4 includes non-volatile storage elements. Examples of such non-volatile storage elements can include magnetic hard discs, optical discs, flash memories, or forms of electrically programmable memories (EPROM) or electrically erasable and programmable (EEPROM) memories.

As illustrated in FIG. 1, the memory 4 can be configured to include health data extraction module 10, machine learning training module 12, efficacy determination module 14, and interaction classification module 16. Modules 10, 12, 14, 16 can take the form of computer-readable instructions that, when executed by control circuitry 6, cause the treatment evaluator 2 to implement functionality attributed herein to modules 10, 12, 14, 16. Though the example of FIG. 1 is described with respect to separate modules 10, 12, 14, 16, it is understood that the techniques described herein with respect to such modules 10, 12, 14, 16 can be implemented in a single module or multiple modules (e.g., two, three, four, etc.) that distribute functionality attributed herein to modules 10, 12, 14, 16 among the multiple modules. In general, memory 4 can store computer-readable instructions that, when executed by control circuitry 6, cause treatment evaluator 2 to operate in accordance with techniques described herein.

The user interface 8 of the treatment evaluator 2 can be configured as an input and/or output device. For example, the user interface 8 can be configured to receive inputs from a data source and/or provide outputs regarding patient health, treatment options, and/or drug interactions, among other information. Examples of the user interface 8 can include one or more of a sound card, a video graphics card, a speaker, a display device (such as a liquid crystal display (LCD), a light emitting diode (LED) display, an organic light emitting diode (OLED) display, etc.), a touchscreen, a keyboard, a mouse, a joystick, or other type of device for facilitating input and/or output of information in a form understandable to users and/or machines.

In operation, the treatment evaluator 2 executes the modules 10, 12, 14, 16 to generate pertinent health data for a patient, to analyze the pertinent health data by a machine learning model to generate predictive health information regarding a future health status of the patient, to generate information regarding interactions between drugs of various drug combinations used to treat morbidities. The treatment evaluator 2 is configured to generate data regarding drugs utilized to treat various morbidities. The treatment evaluator 2 can determine whether particular drug combinations will or will not be effective for a particular patient based on pertinent health data regarding that patient. The pertinent health data is provided to the treatment evaluator 2. For example, the treatment evaluator 2 can execute the health data extraction module 10 to generate the pertinent health data. Electronic medical records of the subject patient can be extracted from electronic medical record (EMR) storage 18 and provided to health evaluator 2. The EMR storage 18 can be a computer-readable database configured to store data regarding electronic medical records of one or more patients. The pertinent health data can be generated based on the EMRs of the patient received from the EMR storage 18.

The treatment evaluator 2 is configured to implement one or more treatment machine learning models to determine the efficacy of drug combinations for treating a subject morbidity (e.g., hypertension, diabetes mellitus, congestive heart failure, etc.). For example, the treatment evaluator 2 can execute the efficacy determination module 14 to generate data regarding the efficacy of a treatment action at treating one or more subject morbidities of the subject patient.

The treatment machine learning models are trained on baseline health data that is generated based on sets of EMRs for each patient in a patient population associated with a subject morbidity. For example, the treatment evaluator 2 can execute the machine learning training module 12 to train the one or more machine learning models. The trained machine learning models can be stored in the memory 4. The machine learning models are trained to predict the efficacy of drug combinations at treating subject morbidities of a patient, to generate data regarding the interactions between drugs in drug combinations, among other health information outputs.

The patient population is a statistically significant sample size of the population. In some examples, the patient population can be each patient within a healthcare system (e.g., hospital, clinic, networks of providers, etc.) that is diagnosed with and treated for the subject morbidity over a sample period. For example, the population size can be 10,000 or more patients, 50,000 or more patients, 100,000 or more patients, 200,000 or more patients, 400,000 or more patients, 600,000 or more patients, etc. In some examples, the population size can be ten times or more larger than the number of drug combinations in the sets of EMRs, providing at least a 10:1 ratio of population size to drug combinations. The sets of EMRs to drug combination ratio can be 20:1, 30:1, 40:1, 100:1, or any other desired ratio suitable for generating statistically significant results. The baseline health data includes information regarding encounters between the patients and the healthcare system, which includes one or more encounters per patient. As such, the baseline health data can include multiple sets of EMRs providing information from multiple encounters for each patient. For example, a ratio of encounters to patients in the patient population can be 1.5:1, 2:1, 3:1, 4:1, or more.

The treatment machine learning model can be configured to determine the efficacy of drugs and drug combinations at treating a subject morbidity for a particular patient and/or across the patient population. The treatment evaluator 2 can identify drug combinations as effective or ineffective for a particular patient based on pertinent health data regarding that particular patient. For example, the treatment evaluator 2 can execute the efficacy determination module 14 to execute one or more of the trained machine learning models and generate information regarding a predicted efficacy of one or more drug combinations at treating the subject morbidity for a particular patient.

The pertinent health data can be based on the particular patient’s EMRs, among other options. The pertinent health data can include various information specific to the patient, such as information regarding current and past morbidities, lifestyle factors (e.g., tobacco use, alcohol use, frequency of exercise, etc.), lab results (e.g., results of blood tests, urine tests, etc.), vital signs (e.g., body temperature, pulse rate, respiration rate, blood pressure etc.), blood oxygen level, height, weight, profession, marital status, age, sex, race, household factors (e.g., number of persons in the household, location such as zip code, etc.), treatment tolerance information, among other pertinent health data. In some examples, the treatment evaluator 2 can be configured to generate at least a portion of the pertinent health data, such as by generating the treatment tolerance information. For example, the treatment evaluator 2 can execute the health data extraction module 10 to generate the pertinent health data.

The treatment evaluator 2 can, by the treatment machine learning model, identify treatments that are anticipated to be effective at treating the subject morbidity for the patient. The treatment evaluator 2 can be configured to generate one or more candidate treatments based on the pertinent health data and the drug combinations identified as effective for the particular patient. For example, the treatment evaluator 2 can execute the efficacy determination module 14 to identify the effective ones of the treatments and generate the candidate treatments.

In some examples, side effect information is generated for the drug combinations, which side effect information includes data regarding side effects associated with particular drugs and/or drug combinations. The treatment evaluator 2 can be configured to identify drug combinations as effective or ineffective for the particular patient based on the side effect information and based on the treatment tolerance information regarding the particular patient. For example, the treatment evaluator 2 can execute the efficacy determination module 14 to identify effective combinations for treating the subject patient. The treatment tolerance information identifies patient-specific side effects that are or are not tolerable for the particular patient. The treatment tolerance information can be generated by the patient, generated by the treatment evaluator 2 based on EMRs of the particular patient, and/or generated by the treatment evaluator 2 based on an analysis of the sets of EMRs for the patient population. For example, the treatment evaluator 2 can execute the health data extraction module 10 to generate the treatment tolerance information. The treatment evaluator 2 can generate one or more candidate treatments based on the drug combinations and associated side effect information that are identified as effective for the particular patient. In some examples, the treatment machine learning model can be trained to identify side effects and combinations thereof that are effective at treating morbidities of a patient. The treatment evaluator 2 can be configured to identify side effects as effective or ineffective at treating the morbidity of a patient and the treatment regimen for that patient can be built based on the side effects or combinations thereof that are identified as effective by the treatment evaluator 2.

In some examples, the treatment evaluator 2 is configured to identify relationships between drugs within drug combinations. For example, the treatment evaluator 2 can execute the interaction classification module 16 to identify and classify relationships between the various drugs forming the drug combinations. The treatment evaluator 2 can identify the efficacy of various drug combinations at treating the subject morbidity. The treatment evaluator 2 can identify the relationships based on comparisons between more effective (e.g., drug combinations having higher efficacy scores) and less effective (e.g., drug combinations having lower efficacy scores) drug combinations, as identified by the treatment machine learning model. The treatment evaluator 2 can identify drugs within a drug combination that negatively and/or positively interact with other drugs of that drug combination based on the efficacy scores and comparisons between various of the drug combinations that have cross-over drugs. Cross-over drugs are drugs that appear in each of the drug combinations being compared.

Treatment evaluator 2 provides significant advantages. Treatment evaluator 2 is configured to generate patient-specific candidate treatments based on the real world information from the sets of EMRs for the patient population. Most diseases can be and are treated with more than one medication and more than one dosage. By analyzing knowledge of all the medication options available, including dosages, as obtained from the sets of EMRs by the treatment machine learning model designed to predict the medication and dosage most likely to control a disease by increasing the likelihood of adherence, the set of medications and dosages that will most likely control one or more diseases for a subject patient can be determined. The treatment evaluator 2 can be a software application that determines the most effective set of medications for an individual patient by determining the set of side effects most likely to be tolerated by each patient. The treatment evaluator 2 can use that information in combination with a database of medication side effects to find the set of medications that will treat the patient’s diseases, and maximize the likelihood that the patient will be prescription adherent. The treatment evaluator 2 can identify the set of side effects least likely to inhibit adherence, which infers the set of medications that is most likely to successfully treat disease. Treatment evaluator 2 can thus provide candidate treatment options for a specific patient that are based on the real world data from the sets of EMRs, providing candidate treatments that are determined to be effective at treating the diseases and that are likely to lead to prescription adherence. Effectively treating the diseases and having prescription adherent patients provides significant savings in both healthcare costs to the individual and the system and prevents unnecessary use of resources to iteratively treat diseases.

The treatment evaluator 2 generates treatment information for a patient based on a wide array of medications and dosages that cannot practically be considered by a medical provider when treating the subject patient. The treatment evaluator 2 can identify and output the best options for treating the patient morbidities and a medical provider can select from the identified options with the knowledge that the treatment information output by the treatment evaluator 2 is identified as effective based on the real world information from historical treatment of the disease, as derived from the EMRs.

Treatment evaluator 2 provides additional advantages for new drug development. If, for example, a drug is being developed that targets an existing disease similar to how other drugs target that disease, it can be assumed that the new drug will be prescribed in similar combinations as the current drugs. Drug interaction data generated by treatment evaluator 2 can be used to guide the design of the new drug such that the drug under development works effectively with the medication combinations that are currently being prescribed. The side effect information generated by treatment evaluator 2 provides additional information for drug development by providing the side effects most and/or least likely to affect prescription adherence, allowing drug development to target the minimization of negative side effects associated with prescription non-adherence.

In some examples, the treatment evaluator 2 can be configured to identify side effects most or least likely to impact prescription adherence. The treatment evaluator 2 can be configured to identify the impactful side effects at various levels of granularity. For example, a general, coarse analysis of the baseline health data can reveal the set of side effects that are least or most likely to inhibit effective treatment for the entire patient population. More specific, finer analysis of subsets of the patient population can reveal the sets of side effects least or most likely to inhibit treatment for subdivisions or cohorts of the patient population. Analysis of an individual’s EMRs can be used to determine the set of side effects least or most likely to inhibit that patient’s treatment.

The side effect information generated for a single morbidity provides information regarding side effect tolerance generally. The side effect tolerance information generated by the treatment evaluator 2 can be utilized to infer the influence of side effects on treatment efficacy, such as which side effects and/or combinations of side effects have an effect on prescription adherence and can thus be considered to have a positive or negative effect on treating disease. The treatment evaluator 2 can thus provide information regarding the influence of side effects and combinations across an array of diseases for which medications are prescribed, without specifically training a machine learning model based on each disease under consideration.

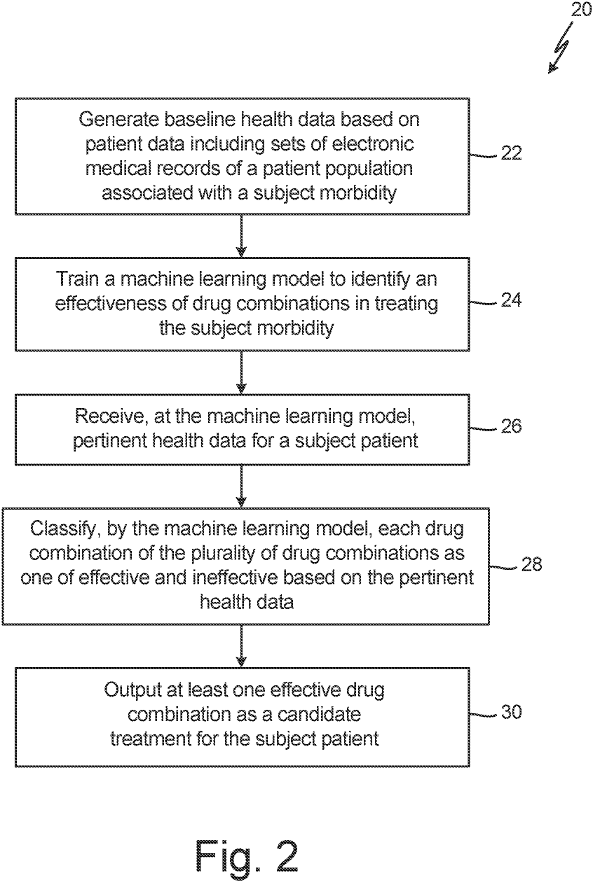

FIG. 2 is a flowchart illustrating method 20 of generating personalized health recommendations. A computing device, such as treatment evaluator 2, can be configured to implement one or more treatment machine learning models trained on baseline health data to generate and output candidate treatment combinations to improve patient outcomes by increasing prescription adherence. The treatment machine learning model is configured to determine the efficacy of various treatment actions at treating one or more morbidities. The computing device is configured to determine treatment actions (e.g., prescribing a drug combination) that patients and providers can take to attain an enhanced (e.g., optimal, in some examples) treatment of the target morbidity for the subject patient.

In step 22, baseline health data is generated based on patient data for patients forming a patient population associated with the subject morbidity. The patient data can include information extracted from sets of EMRs of the patients forming the patient population. Patients can be considered to be associated with the subject morbidity when the patient currently has or previously had a diagnosis of the subject morbidity. For example, a patient can be considered to be associated with the subject morbidity when the set of EMRs for that patient indicate that the patient has or had a diagnosis of that subject morbidity. The sets of EMRs provide information regarding drug combinations that are prescribed for treating the subject morbidity and provide information regarding the result of that treatment protocol (e.g., successful or unsuccessful treatment of the subject morbidity).

In one example, the subject morbidity is hypertension. Hypertension is often treated with multiple medications, such as two to five, from up to nine different medication classes. Each medication class is intended to treat a different aspect of hypertension. Many patients are prescribed multiple medications that can be spread across the different medication classes. In addition, the different drugs can be prescribed at different dosages, providing additional drug combinations in the sets of EMRs. In one set of data, sets of patient EMRs for over 400,000 patients diagnosed and being treated for hypertension were collected. The sets of EMRs were taken over a period of four years and indicated that only about 40% of the patients had their hypertension controlled. Over seventy medications from across the various classes are approved to treat hypertension. Each of the multitudinous number of medications can be prescribed in different combinations of the drugs and in different doses, providing different drug combinations. In the current example, over 10,000 different combinations were prescribed to the 400,000 patients just to treat hypertension. The sets of EMRs revealed that patients were typically prescribed a first drug combination, then a second drug combination (e.g., different drugs, different dosages, a combination of different drugs and dosages, etc.) if the first did not control, then a third drug combination if the second did not control, and so on. The voluminous data, number of drug combinations, and varying treatments indicate that there are too many potential drug combinations for any provider to be able to understand and effectively utilize.

Sets of features are extracted from the sets of EMRs for the patients of the patient population associated with the EMRs. Each set of features is formed based on a patient within the patient population. Each set of features includes patient information, such as information regarding current and past morbidities, lifestyle factors (e.g., tobacco use, alcohol use, frequency of exercise, etc.), lab results (e.g., results of blood tests, urine tests, etc.), vital signs (e.g., body temperature, pulse rate, respiration rate, blood pressure etc.), blood oxygen level, height, weight, profession, marital status, age, sex, race, demographic information (e.g., number of persons in the household, location such as zip code, first language, employment status, etc.), health trends (e.g., test levels associated with the subject morbidity trending up or down, etc.), among other patient information. Each set of features includes drug information regarding the medications prescribed to the patient to treat the one or more morbidities of the patient. In some examples, the drug information can indicate which of the medications are associated with the subject morbidity to treat the subject morbidity. In some examples, the drug information includes information regarding the types of medications prescribed. In some examples, the drug information includes the types of medications and the dosages of the medications prescribed. In some examples, the drug information can include the costs associated with the various drugs, such as out of pocket costs to the patient. It is understood that the drug information can include any desired information regarding the medications prescribed to the patient. In some examples, the set of features includes side effect information, such as indicating that a patient suffered a particular side effect as the result of taking a drug. Each set of features can include tens, hundreds, or thousands of features. For example, each set of features can include greater than or equal to five hundred features, greater than or equal to seven hundred features, greater than or equal to nine hundred features, among other options.

The various features forming each set of features can be extracted directly from the EMRs and/or derived from information contained in the EMRs. For example, lab results, height, weight, body mass index (BMI), etc. can be taken directly from the EMRs to form some of the features in each set of features. Other ones of the features can be derived from the information contained in the EMRs. The set of features for each patient is formed from data that is taken directly from the EMRs and that is derived from the EMRs.

Health trend data can be derived from the information contained in the EMRs. For example, trends in weight, trends in BMI, trends in lab results, trends in systolic blood pressure, trends in diastolic blood pressure, etc. can be determined from the information in the EMRs and utilized to form features within each set of features. The health trend data can include information regarding medications that is derived from the EMRs. For example, trends in the number of medications, such as increasing or decreasing numbers of medication, increasing or decreasing dosages, etc. can be derived from the EMRs.

Health minimum and maximum data can be derived from the information contained in the EMRs. For example, the minimum or maximum values for various health parameters of each patient, such as blood pressure, weight, BMI, lab results, etc., can be derived from the EMRs to form features. The minimum and maximum values can be taken within a certain time period, such as within a time period before treatment began, a time period after treatment began, etc.

Demographic data regarding a patient can be derived from the information contained in the EMRs. For example, the patient’s residence location can be determined from the EMRs and demographic data, such as median income, average household size, etc., can be determined based on census data for that residence location.

Encounter data regarding patient encounters with the healthcare system can be derived from the EMRs. For example, the number and/or frequency and/or type of encounter between the patient and healthcare system can be derived from the EMRs. The encounter data can provide information on how a patient interacts with the healthcare system, such as by phone, in person, etc.

Side effect information can be derived from the EMRs and utilized to form features within each set of features. For example, the EMRs contain information regarding the particular drugs and dosages that are prescribed to each patient. Side effect information for each drug can be generated based on the known side effects associated with each drug, such as that a particular drug is associated with certain side effects such as nausea, dry mouth, inflammation, among others.

Each set of features is labeled as corresponding with either a positive outcome or a negative outcome. The outcome status, positive or negative, is based on the treatment status of the subject morbidity for the patient that the set of features is based on. For example, the subject morbidity being clinically controlled can be labeled as a positive outcome and the subject morbidity not being clinically controlled can be labeled as a negative outcome. The outcomes can be associated with a temporal threshold for controlling the morbidity. The temporal threshold can be associated with the treatment being initiated, such as when drug combinations for treating the subject morbidity are initially prescribed. In such an example, the temporal threshold can begin to run when treatment is initiated for that patient. In some examples, the same set of features is associated with different outcomes depending on the temporal threshold. For example, the sets of EMRs of a first patient of the patient population can indicate that the subject morbidity became controlled at a time fifteen months after treatment began. The set of features extracted from the sets of EMRs of that first patient are associated with a positive outcome for a temporal threshold of at least fifteen months, while that same set of features extracted from the same sets of EMRs of that same first patient is associated with a negative outcome for temporal thresholds less than fifteen months.

The sets of features provide baseline health data for training the treatment machine learning model. Each set of features can be configured as a row of data with the individual features formed in columns. As such, the baseline health data can be configured as a table with each row of data representing a set of features and each column including a feature of that set of features. The treatment machine learning model utilizes machine learning to analyze, understand, and/or respond to patient data. By analyzing a selection of baseline health data, the treatment machine learning model (e.g., decision trees, boosted decision trees, deep learning algorithms, linear regression models, neural networks, confidence assessments, fuzzy logic, among other options) can be trained to recognize, classify, and react to pertinent health data. The machine learning algorithms are computationally complicated and difficult to implement at scale. In some examples, the machine learning model can comprise majority voting among multiple classification machine learning models that together can be considered to form the treatment machine learning model, as discussed in more detail below. The treatment machine learning model can be configured as an ensemble model configured to generate a prediction based on the multiple predictions output by the classification models.

In step 24, the treatment machine learning model is trained on the baseline health data. The treatment machine learning model is trained to identify an efficacy of drug combinations in treating the subject morbidity. In some examples, the treatment machine learning model is trained to make a binary determination as to the efficacy of the drug combinations at treating the subject morbidity. For example, the treatment machine learning model can be trained to classify a drug combination as one of effective and ineffective at treating the subject morbidity for a subject patient. The treatment machine learning model is configured to generate candidate treatment options for treating the subject morbidity for a subject patient.

The baseline health data is split into a first dataset and a second dataset for training the treatment machine learning model. The first dataset can be referred to as training data and the second data set can be referred to as testing data. Each set of features forming the baseline health data are placed in one of the first dataset and the second dataset. The sets of features are randomly assigned to the two datasets such that the sets of features in each of the first dataset and the second dataset are representative of the patient population. As such, each of the first dataset and the second dataset are representative of the patient population as a whole. The first dataset includes more sets of features than the second dataset. In one example, the first dataset is formed by two thirds of the baseline health data and the second dataset is formed by one third of the baseline health data. The first dataset can be formed by 60%, 70%, 80% or another majority percentage of the baseline health data. For example, the first dataset can include a majority of the rows of the baseline health data. The second dataset is formed by the remainder of the baseline health data, such as 40%, 30%, 20%, or another minority percentage of the baseline health data. For example, the second dataset can include a minority of the rows of the baseline health data.

Training of the treatment machine learning model includes an initial training based on the first dataset and testing of that initially trained model based on the second dataset. The treatment machine learning model is initially trained on the first dataset. The labeled sets of features of the first dataset are provided to the treatment machine learning model and the machine learning algorithm is configured to determine a fit to arrive at the labeled conclusion based on the set of features (e.g., to determine whether the subject morbidity is or is not controlled). The treatment machine learning model undergoes supervised learning because the sets of features are labeled with the correct outcome during the training phase of the machine learning model.

The treatment machine learning model can be an ensemble model configured to generate a prediction based on predictions from multiple classification models. The classification models are individual machine learning models (e.g., decision trees, linear regression, etc.) that are individually trained on the baseline health data to generate a prediction regarding patient health (e.g., whether a subject morbidity will or will not be controlled). The multiple classification models together form the treatment machine learning model. The treatment machine learning model can generate a final prediction based on individual predictions made by multiple classification models forming the treatment machine learning model. In a specific example, the treatment machine learning model is formed based on gradient boosted decision trees. The treatment machine learning model can be based on parallel decision tree boosting. In one example, the treatment machine learning model utilizes the XGBoost algorithm during training of the treatment machine learning model.

In a specific example, the treatment machine learning algorithm is provided with the first dataset. The machine learning algorithm is configured to generate decision trees based on the first dataset. Each decision tree can be considered to form a classification model of the treatment machine learning model. In one example, each decision tree is provided with a factor subset that defines the factors that that decision tree considers. For example, if each set of factors includes nine hundred factors, then a first factor subset may contain five hundred of those factors, or another number of those factors. The machine learning algorithm generates a first decision tree based on that first factor subset. A second factor subset may contain another five hundred of those factors, and the factors forming the second factor subset can overlap with one or more of the factors forming the second factor subset. The decision tree is configured such that the decision tree considers only those factors within the factor subset applied to that decision tree. The decision tree can disregard other factors not present within the factor subset of that decision tree. The overlap between the various factor subsets can vary. In some examples, the factor subsets may not overlap. In some examples, the factor subsets may substantially overlap, such as by having 90% or more commonality between factors.

Each decision tree is formed as a series of nodes with bifurcating branches that extend from each node. A prediction (e.g., the subject morbidity is controlled or is not controlled) is made once a terminal node is reached. Each node is configured as if-then-else statements where a first pathway from the node is followed if all of the rules within the node are satisfied and a second pathway from the node is followed if less than all of the rules of the node are satisfied. Each node can be based on one or multiple of the factors forming the factor subset that that decision tree considers.

Parameters of the decision trees are defined prior to the machine learning algorithm constructing the decision trees. Such a process can be referred to as hyperparameter tuning. Hyperparameter tuning involves choosing a set of optimal hyperparameters for the machine learning algorithm prior to the learning process beginning. For example, the hyperparameters regarding a decision tree can define the number of layers forming the tree or the width of the tree. The machine learning algorithm will generate the decision trees based on the first dataset and the hyperparameters. While the hyperparameters are defined, the values of other parameters, such as the weights assigned to various factors (e.g., in a linear regression model) or the weights assigned to various decision trees (e.g., in a gradient boosted decision trees model), are learned during training of the machine learning model.

Each decision tree is iteratively constructed by the machine learning algorithm based on the first dataset and the factor subset for that decision tree. For example, the first decision tree based on the first factor subset can be constructed based on possible decisions for each patient in the first dataset and based on the first factor subset. Each decision tree is formed in a similar manner, but based on different factor subsets. For each decision tree, the machine learning algorithm generates the nodes and the if-then-else statements associated with each node based on the first dataset and the factor subset for that decision tree. It is understood that tens, hundreds, or thousands of decision trees can be generated based on the first dataset.

The multiple decision trees are generated based on the labeled first dataset. After generation, each decision tree is tested based on the second dataset. During testing, the decision trees are provided the unlabeled sets of factors forming the second dataset and the decision trees generate predictions on whether the subject morbidity is or is not controlled. The treatment machine learning model has not been exposed to the second dataset prior to the testing stage. The testing stage provides information on the accuracy of the predictions generated by each decision tree of the machine learning model as the outcome (i.e., whether the subject morbidity is controlled or is not controlled) is already known for each patient forming the second dataset. The predictive accuracy of each decision tree is determined based on the performance of each decision tree at predicting whether the subject morbidity is controlled with regard to the sets of factors forming the second dataset.

The decision trees are weighted based on the predictive accuracy determined from executing each decision tree on the second dataset. A weighting factor is generated for and applied to each decision tree based on the predictive accuracy of each decision tree. More accurate ones of the decision trees, which are the decision trees that had a higher accuracy rate in determining whether the morbidity was or was not controlled in the second dataset, will be assigned a higher weighting factor. More weight is given to the result output by decision trees having higher weighting factors. Less accurate ones of the decision trees will be assigned a lower weighting factor. Less weight is given to the result output by decision trees having lower weighting factors. The weighting factor is based on the accuracy of the prediction for that decision tree across the patient population, as determined based on the predicted outcomes for the second dataset.

The weighted decision trees form the trained treatment machine learning model. During execution of the treatment machine learning model, each decision tree of the treatment machine learning model generates a prediction as to whether the subject morbidity will or will not be controlled based on the subset of factors that each decision tree considers. Each decision tree is configured to determine a probability that the subject morbidity will or will not be controlled. For example, each decision tree can output the probability as a value between zero and one, with prediction values greater than or equal to 0.5 indicating that the subject morbidity will be controlled and prediction values less than 0.5 indicating that the subject morbidity will not be controlled.

The prediction values of all of the decision trees forming the treatment machine learning model are normalized such that the treatment machine learning model generates a prediction value between zero and one. The treatment machine learning model determines that the subject morbidity will be controlled based on the overall prediction value being greater than or equal to 0.5 and determines that the subject morbidity will not be controlled based on the overall prediction value being less than 0.5.

While a specific training example regarding boosted decision trees is discussed, it is understood that the treatment machine learning model can be trained based on any desired machine learning algorithm, such as linear regression algorithms or deep learning algorithms. In some examples, the treatment machine learning model can be trained based on a linear regression algorithm. In such an example, the machine learning algorithm is configured to learn weights applied to each of the factors to minimize an error between the predicted value and a true value. The linear regression equation is shown in Equation 1.

$$Y = w 1 X 1 + w 2 X 2 + w 3 X 3 … + w n X n + B$$

Y is the output variable that is being predicted by the model, such as whether the subject morbidity will or will not be clinically controlled. Each X is a different factor from the set of factors that form the patient data. Each w is a weighting factor for X associated with that weighting factor, and the weighting factors are learned by the treatment machine learning model during training. B is a residual error factor that is also determined by the machine learning model during training. The linear regression model is initially trained based on the information in the first dataset.

In some examples, the treatment machine learning model can be an ensemble model based on multiple linear regression models. Such an ensemble model can be configured similar to the treatment machine learning model based on multiple decision trees. For example, each linear regression model can be configured to make a health prediction based on less than all of the features forming the sets of features. The linear regression models can be assigned weights based on the predictive accuracy of the linear regression models for the second dataset. The treatment machine learning model can generate the health prediction based on a normalized output from each of the multiple linear regression models.

In some examples, multiple sets of baseline health data are generated based on different temporal thresholds and those multiple sets of baseline health data are used to train multiple treatment machine learning models based on the various temporal thresholds. For example, a first treatment machine learning model can be trained based on baseline health data associated with a six month temporal threshold, a second treatment machine learning model can be trained based on baselines health data associated with a twelve month temporal threshold, etc.

The treatment evaluator 2 can be configured to determine the efficacy of various drug combinations at treating the subject morbidity. In some examples, the treatment machine learning model can be configured to generate data regarding the efficacy of the various drug combinations. For example, the treatment machine learning model can be configured to generate efficacy scores for the drug combinations. For example, the treatment machine learning model can generate information regarding a population-wide efficacy of individual drug combinations and can generate the efficacy scores based on the population-wide efficacy. The population-wide efficacy can be determined by the trained treatment machine learning model analyzing the individual drug combinations. For example, the treatment machine learning model can simulate treatment of each patient forming the patient population by each drug combination within the baseline health data. Such a simulation provides information regarding the efficacy of each drug combination at treating the subject morbidity across the patient population as a whole.

The treatment evaluator 2 can analyze individual drug combinations and determine the efficacy of the individual drug combinations relative to the real world efficacy and relative to other drug combinations. For example, the treatment machine learning model can simulate treatment outcomes for the individual drug combinations for each patient of the patient population. In some examples, the trained treatment machine learning model analyzes the individual drug combination across each set of EMRs of the patient population to determine an efficacy of that individual drug combination at treating each patient of the patient population. In such an example, the set of EMRs for each patient forms ranking health data for that particular analysis and the treatment machine learning model determines the efficacy of the drug combination based on that ranking health data. The ranking health data can be considered to form pertinent health data for each such analysis of the individual drug combination across the patient population.

An efficacy score can be generated based on a comparison of an overall efficacy of all drug combinations and the efficacy of the individual drug combination. For example, a first efficacy count for all drug combinations can be derived from the baseline health data, which efficacy count is a count (e.g., number, percentage, etc.) of the patients within the patient population that were effectively treated. The treatment machine learning model can generate additional efficacy counts for individual drug combinations. The additional efficacy counts are based on simulated treatment of each patient in the patient population by the individual drug combinations. For example, the treatment machine learning model can generate a second efficacy count for a first drug combination by simulating treatment of each patient by the first drug combination and determining whether such treatment would be effective. The second efficacy count provides the efficacy of the first drug combination relative to the baseline efficacy provided by the first efficacy count. The baseline, first efficacy count can be compared to the second efficacy count to generate the efficacy score for the first drug combination. For example, the efficacy score can be the difference between the baseline count and the count determined by the treatment machine learning model. The more effective drug combinations will have a higher efficacy score as compared to less effective drug combinations. The efficacy scores provide the relative efficacy of each drug combination at treating the subject morbidity.

In step 26, pertinent health data regarding a subject patient is provided to the treatment machine learning model. The pertinent health data includes patient information regarding the subject patient. The patient information can include, among others, information regarding current and past morbidities, lifestyle factors (e.g., tobacco use, alcohol use, frequency of exercise, etc.), lab results (e.g., results of blood tests, urine tests, etc.), vital signs (e.g., body temperature, pulse rate, respiration rate, blood pressure etc.), blood oxygen level, height, weight, profession, marital status, age, sex, race, household factors (e.g., number of persons in the household, location such as zip code, etc.), health trends (e.g., test levels associated with the subject morbidity trending up or down, etc.). The pertinent health data is received by the treatment machine learning model and analyzed by the treatment machine learning model. In some examples, the pertinent health data is derived, at least in part, from EMRs of the subject patient.

In step 28, the treatment machine learning model analyzes the pertinent health data. The treatment machine learning model determines whether the various drug combinations from the baseline health data would be effective or ineffective at treating the subject morbidity for the subject patient based on the pertinent health data. In some examples, the treatment machine learning model is configured to classify the drug combinations in a binary manner, such that a drug combination is determined to be either effective or ineffective by the treatment machine learning model. In some examples, the drug combinations that have negative efficacy scores, indicating less effective treatment than the real world, baseline efficacy, can be classified as ineffective treatment options.

The treatment machine learning model simulates treatment of the patient by a drug combination to determine whether the drug combination will be or will not be effective at treating the subject morbidity for the patient. For example, each classification model, such as individual logic decision trees, can generate a prediction of whether the drug combination will effectively treat the subject morbidity for the patient. The treatment machine learning model can generate an overall prediction (e.g., the morbidity will or will not be effectively treated) based on the outputs of each of the classification models forming the treatment machine learning model, such as based on the weighted predictions from the multiple logic decision trees. The treatment machine learning model will thus determine whether the drug combination will or will not be effective at treating the subject morbidity for the patient. It is understood that, in some examples, at least some of the classification models may be configured such that that classification model does not consider all of the drugs in the drug combination when making a prediction. For example, the factor subset used to train that classification model may exclude various drugs of the drug combination.

In some examples, the treatment machine learning model is configured to simulate treatment of the patient by each drug combination from the baseline health data. As such, the treatment machine learning model can simulate treatment of the patient by each drug combination derived from the sets of EMRs of the patient population. Simulating treatment of the patient based on each of the drug combinations provides a prediction for each drug combination as to whether that drug combination will or will not provide effective treatment. In some examples, the treatment evaluator 2 can be configured to exclude various of the drug combinations from the simulation based on rules established prior to simulation, such that certain drugs or drug combinations are inappropriate for prescribing certain segments of the patient population.

In step 30, the treatment machine learning model outputs one or more of the drug combinations determined to be effective as candidate treatment options for the subject patient. In some examples, the candidate treatments, as determined by the treatment machine learning model, are output to the user. For example, the candidate treatments can be provided to a provider of the subject patient, such as a doctor, and the provider can select a treatment from among the candidate treatments to prescribe to the subject patient. In some examples, a ranked list of the candidate treatments can be generated and output to the user. The ranked list can be generated based on the efficacy scores of the various drug combinations forming the candidate treatments. For example, the treatment machine learning model can determine which of the various drug combinations would be effective for the subject patient and can then rank those effective ones of the drug combinations based on the efficacy scores generated by the treatment machine learning model. In some examples, effective ones of the drug combinations can be ranked based on the certainty of the prediction generated by the treatment machine learning model. As discussed above, the treatment machine learning model can classify drug combinations as effective based on the prediction value being greater than or equal to 0.5. The drug combinations can be ranked based on the prediction values, such as a first drug combination having a prediction value of 0.85 being ranked higher than a second drug combination having a prediction value of 0.55. In some examples, the effective drug combinations can be ranked based on cost, such as out of pocket cost, from least expensive to most expensive.

Method 20, treatment evaluator 2, and the treatment machine learning model provide significant advantages. Method 20 is configured to generate personalized treatment options for patients. The treatment evaluator 2 can generate personalized treatment information based on pertinent health data regarding the subject patient. The pertinent health data includes personalized information regarding the subject patient. The treatment machine learning model is trained on the baseline health data and generates and outputs effective candidate treatment options based on that personalized information regarding the subject patient. The treatment machine learning model analyzes large volumes of data in a computationally intensive manner. The treatment machine learning model generates and outputs candidate treatment options that are based on the particular attributes of the subject patient. The drug combinations are classified as either effective or ineffective based on the specific attributes of the subject patient. Such personalized healthcare options increase prescription adherence and the likelihood of an effective, desirable outcome for treatment of the subject morbidity in the subject patient. Increasing prescription adherence leads to more effective treatment of the subject morbidity in the populace, decreasing healthcare and other costs associated with unhealthy individuals.

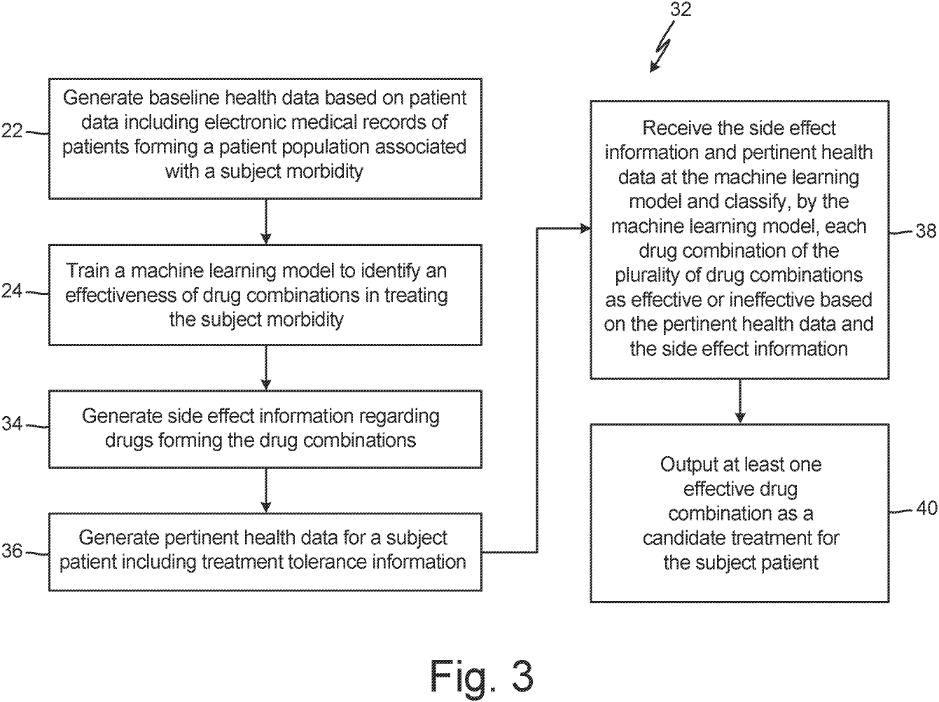

FIG. 3 is a flowchart illustrating method 32 of generating personalized health recommendations to treat a subject morbidity for a subject patient. A computing device, such as treatment evaluator 2 (FIG. 1), is configured to implement one or more treatment machine learning models that are configured to determine the efficacy of drug combinations for treating one or more subject morbidities. Method 32 is similar to method 20 (FIG. 2) in that baseline health data is generated based on sets of EMRs of patients of a patient population associated with a subject morbidity at step 22 and the machine learning model is trained based on the baseline health data at step 24.

The treatment machine learning model is configured to generate candidate treatment options for a subject patient having the subject morbidity. In step 34, side effect information for the drugs forming the drug combinations is generated and associated with the drugs forming the drug combinations. For example, side effect information for each drug can be compiled in an n-dimensional table. The side effect information can be generated based on each individual drug present in the drug combinations. In some examples, a database of side effect information can be generated and stored, such as in memory 4 (FIG. 1) among other computer-readable data storage options. The side effect information can be recalled from the memory 4 and provided to the treatment machine learning model.

In step 36, pertinent health information for a subject patient is generated and provided to the machine learning model. In the example discussed, the pertinent health information includes treatment tolerance information for the subject patient. The treatment tolerance information includes information regarding side effects that can or cannot be tolerated by the subject patient. For example, drugs that cause blurred vision may not be tolerated by patients who need to drive automobiles, drugs that cause weight gain may not be tolerated by more image-conscious patient cohorts, drugs that cause insomnia may not be tolerated by patients with newborn children, etc. With this understanding, the treatment evaluator 2 can predict how different types of side effects can lead to non-adherence in individual patients.

In some examples, the treatment tolerance information can be generated by the patient and then provided to the treatment evaluator 2, such as by a survey or intake form filled in by the patient. In additional or alternative examples, the treatment evaluator 2 can generate the treatment tolerance information based on the EMRs of the subject patient. For example, the treatment evaluator 2 can determine that a patient is less likely to tolerate nausea as a side effect based on the EMRs of the subject patient indicating intolerance for nausea, based on the EMRs indicating non-improvement of morbidities of the subject patient in conjunction with nausea-inducing medications, among other indications.

In step 38, the pertinent health data and the side effect information are received and analyzed by the treatment machine learning model. The drug combinations are classified as effective or ineffective for the subject patient based on the pertinent health data and the side effect information. The pertinent health data includes the treatment tolerance information for the subject patient. The treatment machine learning model is configured to classify the various drug combinations of the baseline health data as effective or ineffective for treating the subject morbidity for the subject patient. The treatment machine learning model can determine that one or more of the drug combinations will be effective at treating the subject morbidity based on the pertinent health data. In some examples, effective ones of the drug combinations, as determined by the treatment machine learning model, can be compared, such as by the treatment evaluator 2, to the side effect information for a first drug combination to determine if a match exists between the treatment tolerance information and side effect information. The first drug combination can be classified as ineffective for treating the subject morbidity based on the comparison indicating a match between the treatment tolerance information and the side effect information.

In some examples, a list of initial candidate drug combinations can be generated by the treatment machine learning model. For example, the list can be formed by those drug combinations determined to be effective by the treatment machine learning model. The list of initial candidate drug combinations can be analyzed based on the side effect information and treatment tolerance data to generate a list of final candidate drug combinations. The determination of effective and ineffective drug combinations is based on the side effect information and treatment tolerance data. The side effect information is associated with each drug of the various drug combinations to provide a side effect status for each drug combination. For example, the side effect information for each drug can be provided to the treatment evaluator 2 in an n-dimensional table that contains the side effect information. The side effect status of the drug combination is the cumulative side effects of the individual drugs forming the drug combination. For example, a drug combination having drugs A, B, and C, with drug A having side effects D, E; drug B having side effects D, F, G; and drug C having side effects D, F will have a side effect status of DDD, E, FF, and G. The side effect statuses of the candidate drug combinations forming the list of initial candidate drug combinations are analyzed based on the treatment tolerance information to generate the list of final candidate drug combinations for the subject patient. For example, assuming that the side effect “D” is nausea and the treatment tolerance information indicates a moderate tolerance for nausea, the drug combination ABC may be classified as ineffective due to the triple nausea effect of that combination. For a patient having treatment tolerance information indicating a strong tolerance for nausea, the drug combination ABC may be classified as effective.

The treatment tolerance information is compared with the side effect information for the initial candidate drug combinations to determine which, if any, of the initial candidate drug combinations satisfy the treatment tolerance requirements of the subject patient. For example, the side effect status of each initial candidate drug combination can be analyzed based on the treatment tolerance information and the initial candidate drug combinations can be classified as effective or ineffective based on that comparison. An initial candidate drug combination can be classified as ineffective based on the potential candidate drug combination including side effects likely to lead to prescription non-adherence, as determined from the treatment tolerance information. An initial candidate drug combination can be classified as effective based on the initial candidate drug combination not including side effects deemed likely to lead to prescription non-adherence, as determined from the treatment tolerance information. In some examples, an initial candidate drug combination can be classified as effective based on the initial candidate drug combination including side effects deemed likely to lead to prescription adherence in the treatment tolerance information. The initial drug combinations determined to be ineffective can be removed as candidate drug combinations to generate the list of final candidate drug combinations. In some examples, the final candidate drug combinations can include each of the initial candidate drug combinations configured in a ranked list, the ranking based on a comparison of the treatment tolerance information and the side effect information. Effective drug combinations having fewer adverse side effects, as indicated by the treatment tolerance information, can be ranked higher than drug combinations having more adverse side effects.

The treatment evaluator 2 can be configured to classify the drug combinations as effective or ineffective based on the side effect information. For example, the drug combinations can be classified as ineffective based on the side effect count meeting or exceeding a threshold count, based on a count of the number of specific side effects (e.g., number of medications sharing a side effect (e.g., nausea)), based on a statistical overrepresentation of specific side effects, etc.

In some examples, the treatment evaluator 2 can be configured to generate a ranked list of final candidate drug combinations based on the side effect information. Analysis of baseline health data and side effect information has shown that more effective ones of the drug combinations tend to have fewer side effects while less effective ones of the drug combinations have more side effects. The treatment evaluator 2 can be configured to rank the candidate drug combinations based on a side effect count of the drug combinations, among other options. For example, the candidate drug combinations having fewer side effects can be ranked higher than candidate drug combinations having more side effects.

In step 40, the final drug combinations that are determined to be effective as candidate treatment options for the subject patient are output. The candidate treatment options can be provided to a provider of the subject patient, such as a doctor, and the provider can select a treatment from among the candidate treatment options as a treatment for the subject patient. In some examples, the ranked list of the candidate treatments can be generated and output to the user, such as based on the efficacy score of the drug combinations, based on the side effect count of the drug combinations, or based on any other desired ranking criteria.

Method 32, treatment evaluator 2, and the treatment machine learning model provide significant advantages. Method 32 is configured to generate personalized treatment suggestions for a patient. It is believed that uncomfortable and/or severe medication side effects contribute to up to 35% of patients being prescription non-adherent. In addition, current technologies focus on the prevention of prescription errors to minimize injury, as opposed to method 32 that provides treatment options based on the most suitable combination of side effects for each patient, leading to prescription adherence to better treat the subject morbidity across the populace. Generating candidate treatment options based on side effect information provides drug combinations that are most likely to be tolerated by the subject patient, leading to increased incidences of prescription adherence. Method 32 identifies effective drug combinations for an individual patient by determining the set of side effects most likely to be tolerated by that patient. Method 32 uses that information in combination with a database of drug side effects to find the set of drugs that will treat the patient’s morbidities and increase the likelihood that the patient will be prescription adherent. Method 32 delivers personalized drug combination recommendations based on predictions of individual patient tolerance for side effects, rather than generic guidelines based on patient populations. Providing the personalized drug combinations for each patient reduces medical expenses associated with prescription non-adherence and associated with continued poor health.

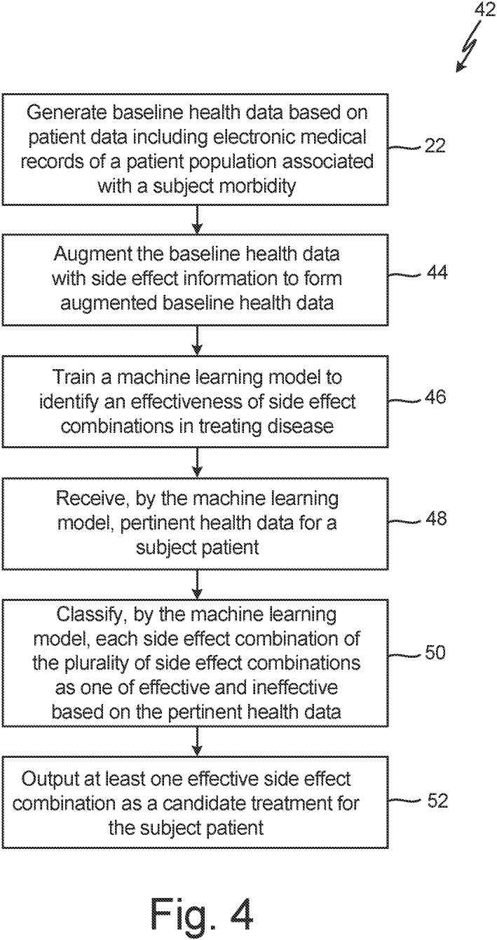

FIG. 4 is a flowchart illustrating method 42 of generating personalized health recommendations to treat morbidities of a subject patient. A computing device, such as treatment evaluator 2 (FIG. 1), is configured to implement one or more treatment machine learning models that are configured to determine the efficacy of drug combinations for treating one or more subject morbidities. Method 42 is similar to method 32 (FIG. 3) and method 20 (FIG. 2) in that baseline health data is generated based on sets of EMRs of patients of a patient population associated with a subject morbidity at step 22.

In step 44, the baseline health data is augmented by side effect information for the various drugs forming the drug combinations present in the sets of EMRs of the patients in the patient population. The baseline health data is augmented by substituting the side effect information for each drug with the drug in the baseline health data, thereby forming augmented baseline health data. The side effect information can be generated based on publicly available information regarding the side effects associated with each drug of the drug combinations. In step 46, the treatment machine learning model is trained to identify an efficacy of various side effects and side effect combinations at treating disease. The treatment machine learning model can be trained similar to the training at step 24, except that the treatment machine learning model is trained based on the augmented baseline health data rather than the baseline health data. The treatment machine learning model is thus trained to determine whether a subject morbidity will or will not be effectively treated based on the side effects associated with treatment of the subject morbidity rather than based on the drugs prescribed to treat the subject morbidity.

In step 48, pertinent health data regarding a subject patient is provided to the treatment machine learning model, similar to step 26. In step 50, the treatment machine learning model analyzes the pertinent health data and classifies the side effect combinations as either effective or ineffective for treatment of the subject patient. The treatment machine learning model determines whether the various side effects and combinations from the baseline health data are effective or ineffective at treating the subject morbidity for the subject patient based on the pertinent health data. In some examples, the treatment machine learning model is configured to classify the side effect combinations in a binary manner, such that a set of side effects is determined to be either effective or ineffective by the treatment machine learning model.

The treatment machine learning model simulates treatment of the patient by a combination of side effects to determine whether the combination of side effects will be or will not be effective at treating the subject morbidity for the patient. For example, each classification model, such as individual logic decision trees, can generate a prediction of whether the combination of side effects will effectively treat the subject morbidity for the patient. The treatment machine learning model can generate an overall prediction (e.g., the morbidity will or will not be effectively treated) based on the outputs of each of the classification models forming the treatment machine learning model, such as based on the weighted predictions from the multiple logic decision trees. The treatment machine learning model will thus determine whether the side effect combination will or will not be effective at treating the subject morbidity for the patient. It is understood that, in some examples, at least some of the classification models may be configured such that that classification model does not consider all of the side effects in a side effect combination when making a prediction. For example, the factor subset used to train that classification model may exclude various of the side effects of a side effect combination.

In some examples, the treatment machine learning model is configured to simulate treatment of the patient by each side effect combination from the augmented baseline health data. As such, the treatment machine learning model can simulate treatment of the patient by each side effect combination derived from the sets of EMRs of the patient population. Simulating treatment of the patient based on each of the side effect combinations provides a prediction for each side effect combination as to whether that side effect combination will or will not provide effective treatment. In some examples, the treatment evaluator 2 can be configured to exclude various of the side effect combinations from the simulation based on rules established prior to simulation, such that certain side effects or side effect combinations are inappropriate for prescribing certain segments of the patient population. For example, blurred vision can be indicated as unavailable for prescribing to a patient employed as a driver.

In step 52, the treatment machine learning model outputs one or more of the side effect combinations determined to be effective as candidate side effect options for the subject patient. In some examples, the candidate side effects, as determined by the treatment machine learning model, are output to the user. For example, the candidate side effects can be provided to a provider of the subject patient, such as a doctor, and the provider can select a treatment from to prescribe to the subject patient based on the candidate side effects. For example, the candidate side effects can be compared to a list of drug side effects to determine a drug combination that satisfies the candidate side effects. In some examples, the treatment evaluator 2 can be configured to generate and output one or more candidate drug combinations based on the candidate side effects generated by the treatment machine learning model. For example, the treatment evaluator 2 can recall drug combinations that correlate to the candidate side effects and output such drug combinations to the provider.

In some examples, the treatment evaluator 2 can generate and output candidate drug combinations based on the drug combinations that best correspond with the candidate side effects. For example, the candidate side effects can be compared to side effect information for the drug combinations from the baseline health data. It is possible that none of the drug combinations will satisfy each of the side effects in the candidate side effects. In such an example, the treatment evaluator 2 can be configured to generate a list of candidate drug combinations based on which of the potential drug combinations best correspond with the candidate side effects. For example, a correlation score can be generated for the drug combinations, which correlation score can be based on both the number of side effects of the drug combination that satisfy the candidate side effects and the number of side effects of the drug combination that differ from the candidate side effects. For example, points can be added to the correlation score for side effects matching the candidate side effects and points can be subtracted from the correlation score for side effects of the side effect combination that are not satisfied and/or for side effects present in the potential drug combination that are not included in the candidate side effect combination.

In some examples, a ranked list of the candidate treatments can be generated and output to the user. In some examples, effective ones of the side effect combinations can be ranked based on the certainty of the prediction generated by the treatment machine learning model. As discussed above, the treatment machine learning model can classify side effect combinations as effective based on the prediction value being greater than or equal to 0.5. The side effect combinations can be ranked based on the prediction values, such as a first side effect combination having a prediction value of 0.85 being ranked higher than a second side effect combination having a prediction value of 0.55. It is understood that the side effect combinations can be ranked based on prediction values among other criteria, such as based on out-of-pocket costs, patient preference, etc. It is further understood that, in some examples, the prediction values can be disregarded in the ranking such that each side effect combination having a ranking greater than or equal to 0.5 is identified as effective. The side effect combinations can be ranked based solely on information other than the prediction values, such as out-of-pocket costs, patient preference, etc.

The treatment machine learning model is configured to generate and output side effect combinations that are predicted to control morbidities for a subject patient based on personalized information for that subject patient. The treatment machine learning model can be configured to predict side effect combinations that will successfully treat diseases of the subject patient even if the treatment machine learning model is not specifically trained on that disease. Instead, knowledge of the candidate side effects can be used to infer that some medication combinations will be ineffective, such as due to prescription nonadherence, while other medication combinations will be effective, due to increasing prescription adherence.

In some examples, the baseline health data can be generated based on multiple different morbidities and whether those morbidities were successfully or unsuccessfully treated. That baseline health data can be augmented based on side effect information for the various drugs in the baseline health data. The treatment machine learning model can be trained to learn the effect of side effect combinations on patient health across the various morbidities, providing generalized information regarding the effect of side effect combinations on patient health.

Method 42, treatment evaluator 2, and the treatment machine learning model provide significant advantages. Method 42 is configured to generate personalized treatment options for patients. The treatment evaluator 2 can generate personalized treatment information based on pertinent health data regarding the subject patient. The pertinent health data includes personalized information regarding the subject patient. The treatment machine learning model is trained on augmented baseline health data and generates and outputs effective candidate side effect combinations based on that personalized information regarding the subject patient. The treatment machine learning model analyzes large volumes of data in a computationally intensive manner. The treatment machine learning model generates and outputs candidate side effects that are based on the particular attributes of the subject patient. The side effect combinations are classified as either effective or ineffective based on the specific attributes of the subject patient. Such personalized healthcare options increase prescription adherence and the likelihood of an effective, desirable outcome for treatment of the subject morbidity in the subject patient. Increasing prescription adherence leads to more effective treatment of the subject morbidity in the populace, decreasing healthcare and other costs associated with unhealthy individuals.

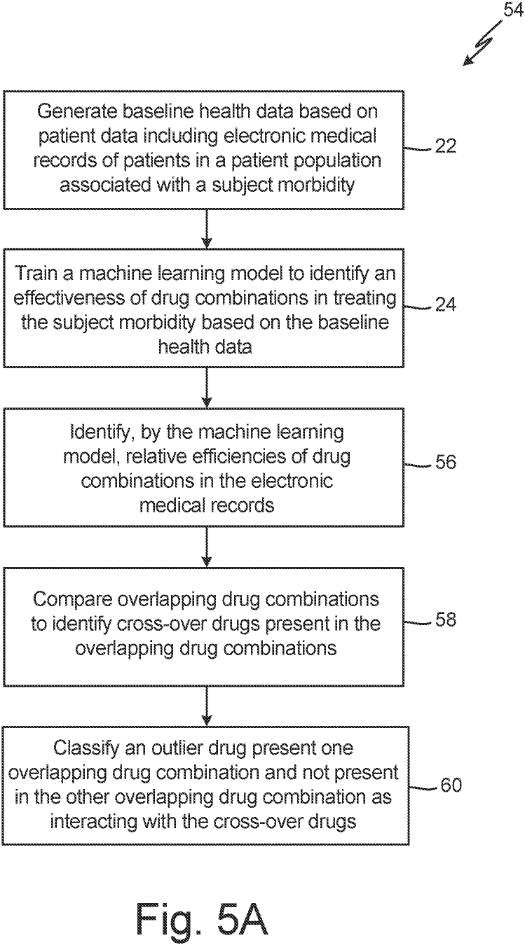

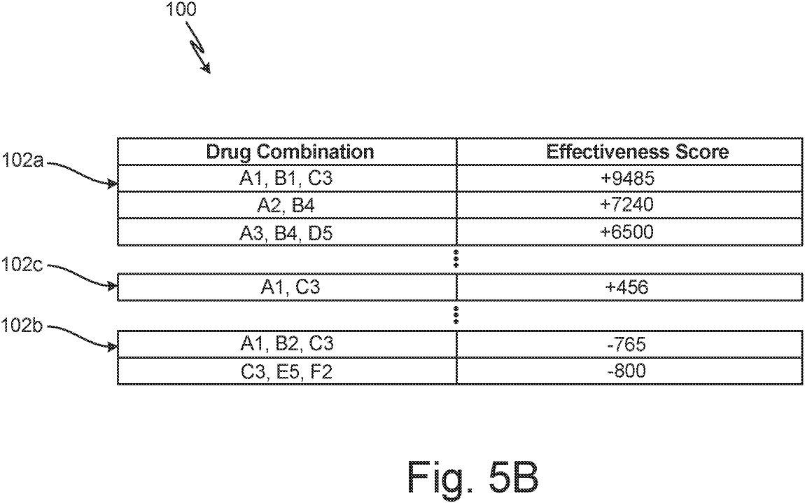

FIG. 5A is a flowchart illustrating method 54 of identifying drug interactions. FIG. 5B illustrates drug interaction ranking table 100. FIGS. 5A and 5B will be discussed together. A treatment machine learning model is configured to identify interactions between the drugs forming various drug combinations used to treat morbidities. A computing device, such as treatment evaluator 2 (FIG. 1), is configured to implement the one or more treatment machine learning models trained on baseline health data to generate and output drug interaction information regarding negative and/or positive interactions between the various drugs forming the drug combinations. Method 52 is similar to method 32 (FIG. 3) and method 20 (FIG. 2) in that baseline health data is generated based on sets of EMRs of patients of a patient population associated with a subject morbidity at step 22 and the treatment machine learning model is trained based on the baseline health data in step 24.

The treatment machine learning model is configured to generate efficacy information regarding the relative efficacy of each drug combination at treating the subject morbidity relative to the baseline, real world efficacy. Drug interaction data regarding interactions between the various drugs within the drug combinations in the baseline health data can be generated based on the efficacy information. In step 44, the treatment machine learning model determines efficacy scores for various of the drug combinations present in the sets of EMRs used to form the baseline health data. In some examples, the treatment machine learning model determines an efficacy score for each of the drug combinations contained in the sets of EMRs. In some examples, the treatment machine learning model is trained to determine the efficacy scores based on the identity of the drugs without considering the associated dosage. In other examples, the machine learning model is trained to determine the efficacy scores based on both drug types and dosages.

The machine learning model can be configured to generate efficacy scores at coarser and finer resolutions. The coarser-resolution efficacy scores can be generated based on an analysis of the patient population as a whole. The coarser-resolution efficacy scores provide information regarding the efficacy of the drug combinations at treating the subject morbidity generally. The finer-resolution efficacy scores can be generated based on an analysis of cohorts of the patient population. The patient population cohorts can be based on any one or more of the features of the sets of EMRs utilized to form the baseline health data. For example, the treatment machine learning model can generate efficacy scores for the various drug combinations at treating male patients older than 55, patients having certain co-morbidities, etc. Generating the finer-resolution efficacy scores provides information regarding drug interactions for subdivisions or cohorts of the patient population. For example, some drug interactions may affect only elderly patients with certain diseases and lab results but such drug interactions may have little to no impact on treatment for other cohorts of the patient population.

The treatment machine learning model is trained on the baseline health data and is configured to generate the efficacy scores based on an analysis of individual drug combinations and the baseline health data. The treatment machine learning model analyzes the individual drug combinations and determines the efficacy of the individual drug combinations by simulating treatment outcomes for the drug combinations. For example, the trained treatment machine learning model can generate a first efficacy score for a first drug combination by simulating treatment by that first drug combination across the sets of EMRs of the patient population. Such an analysis provides an indication of the efficacy of the first drug combination at treating the subject morbidity across the patients for which the simulation was completed. For a coarser-resolution analysis, the drug combination can be analyzed based on each patient of the patient population, providing a population-wide efficacy of that drug combination. For a finer-resolution analysis, the drug combination can be analyzed based on similarly configured cohorts of the patient population. For example, the first drug combination can be analyzed based on EMRs of patients having a certain sex, falling within a certain age range, having certain lab results, among other feature options (e.g., female patients, 55-65 years old, with creatinine levels between 0.6-0.8 milligrams per deciliter (mg/dL)).

In such an example, each set of EMRs can be considered to form ranking health data for that particular analysis and the treatment machine learning model can determine the efficacy of the drug combination based on that ranking health data. The ranking health data can be considered to form pertinent health data for the ranking analysis. The analysis of the first drug combination based on the ranking health data provides information regarding the number of patients in the patient population or cohort of the patient population for which the subject morbidity would be effectively treated by the first drug combination. The efficacy score for the first drug combination can be generated based on a comparison of an initial, baseline efficacy of the various drug combinations contained in the set of EMRs and the determined efficacy for the first drug combination at treating the subject morbidity. The initial, baseline efficacy is based on the real world efficacy of the various drug combinations at treating the subject morbidity, as indicated by the sets of EMRs forming the baseline health data. The baseline efficacy can be determined based on a count of the patients in the patient population for whom the subject morbidity was successfully treated.

For example, the real world, baseline efficacy data indicates that all drug combinations together provide a first efficacy count. The first efficacy count is a count of the number of patients effectively treated, the percentage of patients effectively treated, etc. as derived from the sets of EMRs. The treatment machine learning model simulates treatment of each patient in the patient population based on the first drug combination to generate a second efficacy count for the first drug combination. The first efficacy count is compared to the second efficacy count to generate the efficacy score for that first drug combination. The more effective drug combinations will have a higher efficacy score as compared to less effective ones of the drug combinations. The efficacy score provides the relative efficacy of the drug combinations at treating the subject morbidity. For example, the sets of EMRs forming the ranking health data can indicate that the subject morbidity was effectively treated for 147,000 out of 400,000 patients (36.75% efficacy), and the trained treatment machine learning model can determine that treatment based on the first drug combination would effectively treat the subject morbidity for 153,800 out of the 400,000 patients (38.45%). A comparison of those efficacy scores provides an efficacy score for that first drug combination of +6,800 or +1.7%, with the positive efficacy score indicating that the first drug combination is more effective than the baseline treatment. The treatment machine learning model generates efficacy scores for various of the drug combinations, up to all of the drug combinations from the baseline health data. The efficacy scores provide data regarding the efficacy of the drug combinations at treating the subject morbidity relative to other ones of the drug combinations. As shown in FIG. 5B, the efficacy scores can be used to generate a ranking table 100 for the various drug combinations.

In step 58, the drug combinations are analyzed to identify overlapping ones of the combinations. For example, the treatment machine learning model can be trained to compare the overlapping drug combinations and/or other software or systems can be configured to analyze the outputs from the treatment machine learning model, such as the treatment evaluator 2. Drug combinations are considered to overlap when a drug is present in each of the overlapping drug combinations. A drug that is present in multiple drug combinations is considered to form a cross-over drug between those combinations. For example, in the ranking table 100, the drug combination 102a includes drugs A1, B1, and C3, while the drug combination 102b includes drugs A1, B2, and C3. Drug combinations 102a, 102b overlap because both drugs A1 and C3 are present. Drugs A1 and C3 can be classified as cross-over drugs between the first drug combination 102a and the second drug combination 102b. As shown, drug combination 102a has a relatively high efficacy score while drug combination 102b has a relatively low efficacy score. The differing efficacy scores of the drug combinations 102a, 102b is assumed to be caused by interactions between the various drugs forming the drug combinations 102a, 102b. While a comparison of two drug combinations is discussed, it is understood that any desired number of drug combinations having cross-over drugs can be analyzed and compared to identify interactions between the various drugs forming the drug combinations.

In some examples, overlapping drug combinations are compared based on a difference in the relative efficiencies between the overlapping drug combinations. For example, the most effective drug combinations (e.g., the top 5%, 10%, 15%, 25%, etc. highest efficacy score drug combinations) can be compared to the least effective drug combinations (e.g., the bottom 5%, 10%, 15%, 25%, etc. lowest efficacy score drug combinations) to identify overlapping drug combinations. In some examples, drug combinations having positive efficacy scores, indicating that the drug combination is more effective than the baseline, real world treatment, can be compared to drug combinations having negative efficacy scores, indicating that the drug combination is less effective than the baseline, real world treatment. In such an example, the comparison can be considered to be between effective drug combinations (those having a positive score) and ineffective drug combinations (those having a negative score). It is understood, however, that the drug combinations can be compared in any desired manner to identify overlapping drug combinations.

In step 60, outlier drugs present in the overlapping drug combinations 102a, 102b are classified as interacting with the cross-over drugs. For example, the treatment machine learning model and/or other software or systems can classify the drug interactions, such as classified by the treatment evaluator 2. Outlier drugs are those drugs that are not present in the overlapping drug combinations. In the example shown, the drugs B1 and B2 are outlier drugs with the combination of drugs A1, C3. In the example shown, drug B2 can be classified as negatively interacting with the combination of drugs A1, C3 based on the relatively low efficacy score of drug combination 102b as compared to the efficacy score of drug combination 102a and the baseline efficacy. Similarly, drug B 1 can be classified as positively interacting with the combination of drugs A1, C3 base do the relatively high efficacy score of drug combination 102a as compared to the efficacy score of drug combination 102b and the baseline efficacy.

It is understood that the drug classification can be based on as many or as few drug combinations as desired. In the example shown, drug combination 102c is formed by the combination of drugs A1, C3. The efficacy score of drug combination 102c is intermediate the efficacy scores of drug combination 102a and drug combination 102c. Based on the relative efficacy scores of drug combinations 102a, 102b, 102c, drug B 1, which is present in drug combination 102a but not drug combination 102c, can be classified as positively interacting with the combination of drugs A1, C3, while drug B2, present in drug combination 102b but not drug combination 102c, can be classified as negatively interacting with the combination of drugs A1, C3. Identifying and classifying interactions between drugs does not require any prior information or knowledge regarding adverse interactions between the various drugs. Instead, the treatment evaluator 2 can identify and classify the interactions based on the real world data from the sets of EMRs and based on the efficacy data generated for the drug combinations by the treatment machine learning model.

The drug interaction data generated by method 54 can be used to build a database of drug interaction information, which can be stored in memory 4 (FIG. 1) or in another computer-readable location. The database can provide information about both positive and negative drug interactions. The drug interaction information can be output for use, such as in new drug development and to inform prescription combination decisions by providers.

In a specific example, drug combinations prescribed for hypertensive patients include three types of diuretics: thiazide, loop, and potassium-sparing. The drug interaction data generated for such combinations indicated that combinations of certain loop and potassium-sparing and thiazide and potassium-sparing drug combinations were effective, but certain loop and thiazide drug combinations were not effective. The loop and thiazide combinations are identified and classified as negatively interacting with each other. It was speculated that this is due to the way both loop and thiazide diuretics deplete potassium, and this finding was qualitatively verified by a hospital pharmacist intimately familiar with treating hypertensive patients.

Method 54, treatment evaluator 2, and the machine learning model provide significant advantages. The drug interaction data generated by method 54 can be used to guide current and future drug development. Some medication combinations have been shown to be effective in treating the subject morbidity. Similar but slightly different combinations have been shown to be counterproductive in treating the subject morbidity. Method 54 can identify those drugs that cause the negative and/or positive interactions, providing understanding as to the cause of the interactions and effective/ineffective treatment. Drug development can be guided by this understanding such as, for example, to design new drugs that work better with popular combinations. Such understanding is valuable in drug development by identifying interactions that may not be apparent from closely controlled clinical trials of various drugs. Further, method 54 can predict effective and/or ineffective drug combinations about which little is known, such as when new medications are approved. It is understood that method 54 is not confined to drug combinations prescribed to treat a single disease like hypertension, nor is method 54 limited to combinations consisting of two drugs. The interactions of any number of drugs forming the drug combinations can be identified by method 54.

Current knowledge of medication interactions is limited to interactions identified in clinical trials which are limited to relatively small patient populations and small sets of medication combinations. Method 54 considers a statistically significant number of EMRs, such as hundreds of thousands or multiple millions of EMRs in some examples, and can identify interactions between drugs forming many drug combinations, such as tens of thousands of drug combinations. In effect, method 54 can be used to conduct a virtual clinical trial on patient EMRs by analyzing patient health data collected on a statistically significant number of patients, such as tens or hundreds of thousands or millions of patients, over multiple years. Method 54 can generate a database of drug interactions, both positive and negative, that provides prescription decision support for individual patients.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the present disclosure.

1. A method of identifying drug interactions, the method comprising:
generating, by a machine learning model trained to identify an efficacy of a plurality of drug combinations in treating a subject morbidity based on baseline health data and implemented on a treatment evaluator having a memory and control circuitry, efficacy data for each drug combination of the plurality of drug combinations, wherein the baseline health data is generated based on set of features extracted from sets of electronic medical records of each patient of a patient population associated with the subject morbidity and labeled as corresponding to either a positive outcome or a negative outcome with respect to the subject morbidity;
comparing a first drug combination of the plurality of drug combinations to a second drug combination of the plurality of drug combinations to identify a cross-over drug present in both the first drug combination and the second drug combination; and
classifying a first drug present in the second drug combination and not present in the first drug combination as interacting with the cross-over drug present in both the first drug combination and the second drug combination, wherein the efficacy data indicates that the first drug combination has a different efficacy than the second drug combination.

2. The method of claim 1, wherein generating, by the machine learning model, efficacy data for each drug combination of the plurality of drug combinations comprises:
generating, by the machine learning model, an efficacy score for each drug combination of the plurality of drug combinations.

3. The method of claim 2, wherein generating, by the machine learning model, the efficacy score for each drug combination of the plurality of drug combinations comprises:
simulating, by the machine learning model, treatment of each patient in the patient population by each drug combination of the plurality of drug combinations to generate an individual efficacy count for each drug combination of the plurality of drug combinations.

4. The method of claim 3, wherein generating, by the machine learning model, the efficacy score for each drug combination of the plurality of drug combinations further comprises:
comparing a baseline efficacy count with the individual efficacy count, the baseline efficacy count generated based on the sets of electronic medical records for the patient population; and
generating the efficacy score for each drug combination of the plurality of drug combinations based on a difference between the individual efficacy count and the baseline efficacy count.

5. The method of claim 4, further comprising:
classifying a drug combination having a negative individual efficacy score as ineffective for treating the subject morbidity.

6. The method of claim 2, further comprising:
compiling the efficacy scores for each drug combination into a drug interaction table.

7. The method of claim 2, further comprising:
ranking each drug combination of the plurality of drug combinations based on the efficacy scores.

8. The method of claim 1, wherein the baseline health data does not include drug interaction data.

9. The method of claim 1, wherein a ratio of the patients forming the patient population to the plurality of drug combinations is at least 10:1.

10. The method of claim 1, wherein the sets of electronic medical records include at least 100,000 sets of electronic medical records.

11. The method of claim 1, wherein training the machine learning model to identify an efficacy of a plurality of drug combinations in treating the subject morbidity based on the baseline health data includes:
dividing the baseline health data into a first dataset and a second dataset;
initially training the machine learning model on the first dataset;
testing the initially trained machine learning model on the second dataset.
building a plurality of classification models during the initial training; and
generating weights for each classification model of the plurality of classification models during the testing to generate a plurality of weighted classification models, the weights based on an accuracy of each classification model at predicting a correct outcome for the second dataset;
wherein the machine learning model is configured to generate a prediction based on predications from the plurality of weighted classification models.

12. The method of claim 11, wherein building the plurality of classification models during the initial training comprises:
generating a plurality of feature subsets from the sets of features; and
building each classification model based on a feature subset of the plurality of feature subsets.

13. The method of claim 1, wherein the machine learning model includes an ensemble classifier configured to classify each drug combination as effective or ineffective based on predictions from a plurality of classification machine learning models.

14. The method of claim 13, wherein the plurality of classification machine learning models are formed as one of decision trees, linear regression models, and deep learning algorithms.

15. The method of claim 1, further comprising:
identifying a third drug combination of the plurality of drug combinations that contains only the cross-over drug present in the first drug combination and the second drug combination;
comparing a first efficacy score for the first drug combination to a third efficacy score of the third drug combination; and
classifying an outlier drug present in the first drug combination and not in the third drug combination as positively or negatively interacting with the cross-over drug based on the comparison between the first efficacy score and the third efficacy score.

16. The method of claim 15, further comprising:
classifying the outlier drug as positively interacting with the cross-over drug based on the comparison of the first efficacy score and the third efficacy score indicting that the first drug combination is more effective at treating the subject morbidity than the third drug combination.

17. The method of claim 15, further comprising:
classifying the outlier drug as negatively interacting with the cross-over drug based on the comparison of the first efficacy score and the third efficacy score indicting that the first drug combination is less effective at treating the subject morbidity than the third drug combination.

18. The method of claim 15, further comprising:
comparing a second efficacy score for the second drug combination to the third efficacy score of the third drug combination; and
classifying the first drug as positively or negatively interacting with the cross-over drug based on the comparison between the second efficacy score and the third efficacy score.

19. The method of claim 1, further comprising:
classifying, by the treatment evaluator, the first drug as positively or negatively interacting with the cross-over drugs based on the comparison of the efficacy data; and
outputting, by the treatment evaluator, the classification of the first drug.

20. A method of identifying drug interactions, the method comprising:
generating, by a machine learning model trained to identify an efficacy of a plurality of drug combinations in treating a subject morbidity based on baseline health data and implemented on a treatment evaluator having a memory and control circuitry, efficacy data for each drug combination of a plurality of drug combinations present in the baseline health data for treating the subject morbidity, wherein the baseline health data is generated based on sets of features extracted from sets of electronic medical records of each patient of a patient population associated with the subject morbidity and labeled as corresponding to either a positive outcome or a negative outcome with respect to the subject morbidity;
identifying, by the machine learning model, a first subset of drug combinations of the plurality of drug combinations as effective and a second subset of drug combinations of the plurality of drug combinations as ineffective;
comparing the first subset of drug combinations to the second subset of drug combinations to identify cross-over drugs present in both a first drug combination of the first subset of drug combinations and a second drug combination of the second subset of drug combinations;
classifying a first drug present in the second drug combination and not present in the first drug combination as adversely interacting with the cross-over drugs present in both the first drug combination and the second drug combination; and
outputting, by the treatment evaluator, interaction data regarding the first drug, the interaction data indicating that the first drug adversely interacts with the cross-over drugs.
